(19) United States
(12) Patent Application Publication
(10) Pub. No.: US 2006/0141013 A1
(43) Pub. Date: Jun. 29, 2006

(54) Lysine mixture and apparatus and method for administering lysine

(76) Inventor: Jennifer L. Welser, Palo Alto, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/360,328

(22) Filed: Feb. 22, 2006

Related U.S. Application Data
(62) Division of application No. 10/290,586, filed on Nov. 7, 2002, now abandoned.

(51) Int. Cl. A61K 31/198; A61K 36/48; A23K 1/165; A23K 1/17

(52) U.S. Cl. 424/442; 424/757; 514/564

(57) ABSTRACT

A mixture is described comprising L-lysine and a base substance palatable to cats. Also described is a improved method and apparatus for administering L-lysine using an oral syringe.

This application is a divisional application of U.S. patent application Ser. No. 10/290,586, filed Nov. 7, 2002.

### BACKGROUND

1. Field of the Invention

This invention relates generally to the field of veterinary medication. More particularly, the invention relates to an improved L-lysine mixture and an apparatus and method for administering L-lysine to a pet.

2. Description of the Related Art

Feline herpesvirus type I (“FHV-1”) is a common viral infection in cats which can affect the eyes and respiratory tract. Most cats are initially infected as kittens and a significant number of cats become chronic carriers without displaying evidence of clinical disease. In a recent study, 97% of cats tested positive for an antibody reaction to FHV-1, 95% of which were higher than the vaccine alone would elicit. The most common signs of FHV-1 include upper respiratory infection and ocular diseases such as conjunctivitis and corneal ulceration.

One strategy to assist in the treatment of FHV-1 identified in the last few years is the oral administration of L-lysine. It is hypothesized that L-lysine inhibits herpesvirus growth through competitive inhibition of arginine, which is required for viral multiplication. Recent reports suggest that maintenance of cats on L-lysine will result in a longer period of time between ocular viral flare-ups. Moreover, if signs of the virus reoccur, the infection is typically milder and of a shorter duration. L-lysine is typically administered to cats at a dosage of 250-1000 mg daily.

L-lysine supplements have been prescribed for humans for many years to reduce the effects of Human Herpes Simplex 1 (“HSV-1”). It is estimated that between 60% and 90% of humans have HSV-1, the most common clinical sign of which is a fever blister around the mouth. Current over-the-counter L-lysine formulations prescribed for human treatment include tablets, capsules, gel caps and powders.

To treat FHV-1, cat owners are currently expected to purchase and administer one of the over-the-counter L-lysine preparations designed for human consumption. There are several problems with this scenario. Cats are not amenable to “pilling” or forced liquids, particularly on a long term basis. Moreover, mixing medications such as L-lysine with food provides for unreliable dosing, particularly in households with multiple cats.

Accordingly, what is needed is an improved apparatus and method for administering L-lysine to cats and/or other pets. What is also needed is a mixture of L-lysine and a base substance which is palatable to cats and/or other pets.

### SUMMARY

A mixture is described comprising L-lysine and a base substance palatable to cats. Also described is a improved method and apparatus for administering L-lysine using an oral syringe.

### BRIEF DESCRIPTION OF THE DRAWINGS

A better understanding of the present invention can be obtained from the following detailed description in conjunction with the following drawings, in which:

FIG. 1 illustrates one embodiment of an apparatus for orally administering L-lysine to a cat or other pet.

### DETAILED DESCRIPTION

In the following description, for the purposes of explanation, numerous specific details are set forth in order to provide a thorough understanding of the present invention. It will be apparent, however, to one skilled in the art that the invention may be practiced without some of these specific details.

In one embodiment, L-lysine is mixed into a flavored gel, paste or other base substance known to be palatable to cats. Different flavored base substances may be used to achieve the desired effect. A malt or fish flavor has been found to be particularly suitable for cats. However, various different flavorings may be used while still complying with the underlying principles of the invention including, but not limited to, beef, chicken, liver, lamb, turkey, cheese, duck, shrimp, crab, tuna, salmon, seafood, whitefish, sardine, cod, and catfish.

The base may be created from various substances including, but not limited to, malt syrup, corn syrup, soybean oil, cod liver oil, cane molasses, glycerin, gelatin byproducts, methylcellulose, digest of poultry and tuna by-products, safflower oil, barley malt syrup, white petrolatum & light mineral oil and/or any combination thereof. In one embodiment, the following specific base ingredients are used (listed in order of decreasing concentration): corn syrup, malt syrup, soybean oil, water, cane molasses, methylcellulose, and sodium benzoate (preservative). In another very specific embodiment, the palatable base is comprised of 47% corn syrup, 30% malt syrup, and 23% liquid petroleum. It should be noted, however, that the underlying principles of the invention are not limited to any particular set of palatable base substances or any particular concentration thereof.

L-lysine may also be added to other known remedies such as hairball remedies and vitamin supplements. For example, in one embodiment, the base substance, in addition to containing palatable compounds, includes elevated percentages of liquid petroleum, glycerine, light mineral oil, and/or any other compounds known to aid cats in expelling hairballs. In one specific embodiment, the following components are used for the base, in the following proportions: 47% malt syrup, 44% liquid petroleum, 7% glycerine, 2% Acacia and less than 0.5% of Vitamin B1.

The L-lysine used in the mixture may be a raw form of L-lysine such as, for example, an L-lysine powder (e.g., 78-99% pure with 85%<1 mm granulation size). In one embodiment, the L-lysine used in the mixture is 2,6-Diaminohexanoic acid monohydrochloride (C6H14N202.HCL). Different forms of L-lysine may be used while still complying with the underlying principles of the invention.

Various different concentrations of L-lysine may be used in the mixture. In one embodiment, L-lysine is initially dissolved in water at a concentration of 500 mg/ml. The L-lysine/water solution is then mixed with the palatable base substance at a 1:1 ratio (i.e., 50% solution and 50% palatable base), resulting in an L-lysine concentration of 250 mg/ml. Given that the current recommended dosage of L-lysine is 250 to 1000 mg daily, two 2 ml doses may be administered daily. Alternatively, a single 4 ml dose may be administered (if the cat is willing to consume the 4 ml dose). It should be noted, however, that the underlying principles of the invention are not confined to any particular concentration of L-lysine or any particular dosage. For example, any concentration up to the maximum amount capable of being dissolved in water may be used (e.g., 64.2 gm/100 ml water @ 20□ C, or approximately 650 mg/ml). Moreover, the L-lysine solution may then be added to the palatable base at various different ratios, based on the desired L-lysine concentration and/or the desired consistency of the final mixture (e.g., 2:1, 3:1, 1:2, . . . etc).

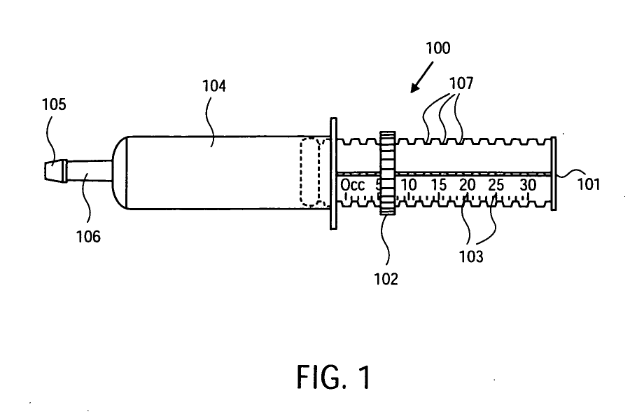

In one embodiment, the mixture containing L-lysine is packaged and/or administered with an oral syringe 100 such as the one illustrated in FIG. 1. The oral syringe 100 includes a storage chamber 104 for storing the L-lysine/base mixture and a plunger 101 cooperatively engaged with the chamber 104 to force a specified amount of the L-lysine mixture out through the dispensing end 106 of the chamber 104. In operation, a cat licks the L-lysine mixture as it is pushed out through the dispensing end 106 of the chamber 106. A cap 105 is fixedly engaged over the dispensing end 106 of the chamber 104 when the oral syringe 100 is not being used.

In one embodiment, a special type of oral syringe, sometimes referred to as a “dial-a-dose” oral syringe is used to administer the L-lysine mixture. As illustrated in FIG. 1, the plunger 101 of this type of syringe includes a dose dialing knob 102 for selecting a specified amount of the L-lysine mixture. The internal surface of the dose dialing knob 102 includes a track which engages with a series of grooves 107 etched on the surface of the plunger 101. As the dose dialing knob 102 is rotated in a clockwise direction it moves lengthwise along the plunger 101 towards the dispensing chamber 106; as the dose dialing knob 102 is rotated in a counter-clockwise direction it moves lengthwise along the plunger 101 away from the dispensing chamber 106.

In operation, the user rotates the dose dialing knob 102 with a counter-clockwise rotation to move the dose dialing knob 102 along the plunger 101, away from the dispensing chamber 104. Volume markings 103 on the plunger indicate the volume of the L-lysine mixture associated with the distance moved by the dialing knob 102 along the plunger. When the dialing knob 102 is positioned at the desired volume marking (e.g., indicating 500 mg of L-lysine for a cat), the user may then push the plunger into the chamber, dispensing the desired amount of L-lysine. When the correct amount of L-lysine has been dispensed, the dose dialing knob 102 is positioned directly adjacent to the dispensing chamber 104, thereby preventing the plunger from being pushed further into the dispensing chamber 104.

Throughout the foregoing description, for the purposes of explanation, numerous specific details were set forth in order to provide a thorough understanding of the invention. It will be apparent, however, to one skilled in the art that the invention may be practiced without some of these specific details. For example, while the embodiments described above focus on the treatment of cats, the invention may be used to treat various other animals. Moreover, while the embodiments described above mention specific components used to create a palatable base, and specific concentrations of L-lysine, the underlying principles of the invention are not limited to any particular palatable base composition or any particular L-lysine concentration. Finally, while the embodiments described above focus on dispensing L-lysine with a dial-a-dose oral syringe, various alternate dispensing mechanisms may be employed. Thus, the scope and spirit of the invention should be judged in terms of the claims which follow.

1-23. (canceled)

24. An apparatus comprising:
an oral syringe having a dispensing chamber and a plunger; and
a mixture comprising L-lysine and one or more substances palatable to cats contained within the dispensing chamber.

25. The apparatus as in claim 24 wherein the oral syringe is a dial-a-dose oral syringe.

26. The apparatus as in claim 24 wherein the concentration of the L-lysine in the mixture is 250 to 2000 mg/ml.

27. The apparatus as in claim 24 wherein the substance palatable to cats comprises malt syrup.

28. The apparatus as in claim 24 wherein the substance palatable to cats comprises corn syrup.

29. The apparatus as in claim 24 wherein the substance palatable to cats comprises soybean oil.

30. The apparatus as in claim 24 wherein the substance palatable to cats comprises cod liver oil.

31. The apparatus as in claim 24 wherein the substance palatable to cats comprises cane molasses.

32. The apparatus as in claim 24 further comprising a substance added to the mixture to aid cats in expelling hairballs.

33. The apparatus as in claim 32 wherein the substance to aid in expelling hairballs comprises liquid petroleum, glycerine, or light mineral oil.

34. The apparatus as in claim 24 further comprising one or more vitamins added to the mixture.

35-41. (canceled)

42. A method of treating a cat, the method comprising:
observing a condition of the cat;
administering to the cat, through a syringe, after the observing, a mixture comprising L-lysine and a base substance which is palatable to cats, wherein the mixture is palatable to cats and has a consistency such that the mixture is dispensable and extrudable through a dispensing end of the syringe and into a mouth of the cat and wherein the concentration of L-lysine is from about 250 to 2000 mg/ml.

43. A method as in claim 42 wherein the base substance comprises at least one of malt syrup; corn syrup; soybean oil; cod liver oil; cane molasses; liquid petroleum; glycerine; and light mineral oil, and wherein the method treats the cat for an infection of feline herpesvirus type I.

44. A method as in claim 42 wherein the syringe is used to measure out a dosage which is packaged in the syringe, and wherein the mixture has a consistency of a paste.

45. A method as in claim 44 wherein the syringe is a dial-a-dose oral syringe which includes a plunger and wherein the cat licks the syringe during the administering.

46. A method as in claim 44 wherein the ratio of L-lysine to the base substance is between 3:1 and 1:3.

47. A method of treating a cat, the method comprising:
observing a condition of the cat;
administering orally to the cat, after the observing, a mixture comprising L-lysine and a base substance which is palatable to cats, wherein the mixture is palatable to cats.

48. A method as in claim 47 wherein the concentration of L-lysine in the mixture is from about 250 to 2000 mg/ml.

49. A method as in claim 48 wherein the condition is an eye condition and the mixture has a consistency of a paste.

50. A method as in claim 49 wherein the administering is performed through an opening of a chamber into which the mixture is packaged.

51. A method as in claim 50 wherein the cat licks the mixture during the administering.

52. A method of treating a cat, the method comprising:
positioning a dispensing end of a chamber near a mouth of the cat;
administering orally to the cat a mixture comprising L-lysine and a base substance which is palatable to cats, wherein the mixture is palatable to cats and has a consistency such that the mixture is dispensable and extrudable through the dispensing end into the mouth of the cat and wherein the mixture is pushed out by force through the dispensing end and wherein the concentration of L-lysine is therapeutically sufficient to treat Feline herpesvirus type I (FHV-1).

53. A method as in claim 52 wherein a user pushes a specified amount of the mixture out of the dispensing end and the specified amount is a predetermined dosage to treat FHV-1.

54. A method as in claim 53 wherein the concentration of L-lysine is from about 250 to 2000 mg/ml.

55. A method as in claim 53 wherein the mixture has a consistency of a paste and wherein the mixture is packaged in the chamber.
